(19) United States
(12) Patent Application Publication
(10) Pub. No.: US 2024/0001047 A1
(43) Pub. Date: Jan. 4, 2024

(54) INFUSION LIQUID HEATING AND FLOW-VELOCITY-MONITORING SYSTEM FOR CLINICAL USE

(71) Applicant: DALIAN UNIVERSITY OF TECHNOLOGY, DALIAN CITY (CN)

(72) Inventors: KAI-RONG QIN, DALIAN CITY (CN); HSIANG-CHEN CHUI, DALIAN CITY (CN); YING XU, DALIAN CITY (CN); ZHI-YUAN WANG, DALIAN CITY (CN); YI-TENG WANG, DALIAN CITY (CN)

(21) Appl. No.: 18/339,142

(22) Filed: Jun. 21, 2023

(30) Foreign Application Priority Data
Jul. 4, 2022 (CN) 202210776851.X

(51) Int. Cl. A61M 5/44; G01F 1/688

(52) U.S. Cl. CPC A61M 5/44; G01F 1/688

(57) ABSTRACT

An infusion liquid heating and flow-velocity-monitoring system for clinical use is provided. The system includes a dynamic heating module, a measurement and analysis module for average velocity in infusion tubes, a velocity adjustment module, an alarming and automatic clipping module, a mobile-phone-computer remote monitoring module, an operable shared module, and a micro control module. A method of determining average velocity on a cross section of a tube according to a solution to Fourier series in a dynamic thermal dispersion equation of a steady flow in the tube is provided. Moreover, functions including monitoring infusion rate, automatic heating, monitoring whether infusion fluid in the infusion tube is empty, and automatic clipping and closing of the tube for preventing blood backflow, etc. are achieved. Lastly, specific applications developed which is connected with mobile phones by 5G network, Bluetooth and WIFI are used to display status of the infusion fluid.

### BACKGROUND OF THE INVENTION

### Field of the Invention

The present invention relates to an infusion liquid heating and flow-velocity-monitoring system for clinical use, especially to an infusion fluid heating and flow-monitoring system based on principles, methods and techniques related to fluid mechanics, heat transfer, signals and systems, dynamic heating, infrared temperature sensing, automatic control, etc. in the field of clinical medical techniques.

### Description of Related Art

In clinical treatment, infusion is a common way for medication via intravenous application and health care staff often need to monitor infusion process closely for dealing with certain conditions such as a new bag with infusion fluid is attached to replace the old one when the infusion fluid runs out, whether infusion rate is suitable, blood backflow in an infusion tube caused by accidental stop of the infusion process. Moreover, in winder of cold areas, lower temperature of the infusion fluid makes patients feel uncomfortable. Thus there is a need to develop a small-scale device or system clipped on the infusion tube not only able to automatically clipping and closing the infusion tube for prevention of blood backflow and heating the infusion fluid to suitable constant temperature, but also used for monitoring the infusion rate, whether the infusion fluid/medicine runs out and timely sending state of the infusion fluid back to mobile phones of the health care staff for alarming in advance. Thereby the health care staff can monitor the infusion process conveniently.

There is a plurality of infusion fluid monitoring devices available on the market now. Refer to literature, Xin Zeng, Xi-Yao Liang, Ying Zhou, design and application of smart infusion alarm system based on fluid level monitoring. journal of Medical Higher Vocational Education and Modern Nursing, 2022, 5(03):262-266, a specific fluid level in patient's infusion bottle is monitored by potential changes of an infrared receiving tube while an alarm is issued by three-color warning lights and buzzers and information related to patient's infusion fluid is sent back to medical staff by wireless network. Refer to Chinese Invention Patent No. CN211096680U “a drip alarm for infusion fluid monitoring” applied by Yun Lu, Wen-Jing Li, Hao Kou, Qiao-Li Jiang in 2020, drip status is checked by infrared emitting and receiving module. When an abnormal situation occurs during infusion process, an audible and visual alarm reminds medical staff and a drip tube is clipped tightly by an elastic stopper.

The above devices available now use infrared to monitor whether liquid medicine in the infusion tube is empty and issue an alarm by audio signals or through connection with mobile phones by Bluetooth. A function of automatic clipping and closing is also provided. However, these devices don't provide automatic heating function and flow rate monitoring which makes patients and health care staff unable to predict end time of the infusion. Thus there is room for improvement and there is a need to provide a new device which not only has functions of flow rate monitoring and automatic heating of the infusion fluid, but also monitors whether the liquid medicine in the infusion tube is empty and automatically clips for closing the infusion tube and prevention of blood backflow. At the same time, the device can be connected with mobile phones by 5G network, Bluetooth, WIFI, etc. and specific applications has been developed to display status of the infusion fluid and monitor the infusion fluid.

As to the function of flow rate detection of the infusion tube, common ultrasonic Doppler flow sensing technique, laser Doppler Flow measurement technique, and magnetic inductive flow sensing technique are unable to meet this requirement due to certain reasons such as lack of micro-particles in liquid infused, high cost, etc. Based on principles of fluid mechanics, heat transfer, and signals and systems, the present system provides a method of determining average velocity in a cross section of a tube according to a solution to Fourier series in a dynamic thermal dispersion equation of a steady flow in a cylindrical tube. Moreover, functions including monitoring infusion rate, automatic heating, monitoring whether liquid medicine in the tube is empty, and automatic clipping and closing of the tube for prevention of blood backflow, etc. are achieved by principles, methods, and techniques related to dynamic heating, infrared temperatures, and automatic control, etc. Lastly, the system connected with mobile phones by 5G network, Bluetooth, WIFI, etc. and specific user's end developed is used to display status of the infusion fluid monitored.

### SUMMARY OF THE INVENTION

Therefore, it is a primary object of the present invention to provide an infusion liquid heating and flow-velocity-monitoring system for clinical use based on principles, methods and techniques related to fluid mechanics, heat transfer, signals and systems, dynamic heating, infrared temperature sensing, automatic control, etc. Based on principles of fluid mechanics, heat transfer, and signals and systems, the system provides a method of determining average velocity on a cross section of a tube according to a solution to Fourier series in a dynamic thermal dispersion equation of a steady flow in a cylindrical tube. Moreover, functions including monitoring infusion rate, automatic heating, monitoring whether liquid medicine in the tube is empty, and automatic clipping and closing of the tube for preventing blood backflow, etc. are achieved by principles, methods, and techniques related to dynamic heating, infrared temperatures, and automatic control, etc. Lastly, specific user's end developed which is connected with mobile phones by 5G network, Bluetooth, WIFI, etc. are used to display status of infusion liquid monitored.

In order to achieve the above objects, an infusion liquid heating and flow-velocity-monitoring system for clinical use according to the present invention includes a dynamic heating module, a measurement and analysis module for average velocity in infusion tubes, a velocity adjustment module, an alarming and automatic clipping module, a mobile-phone-computer remote monitoring module, an operable shared module, and a micro control module.

The dynamic heating module consists of heating sources and a driving circuit for the heating sources. The two heating sources are fixed on two sides of an infusion tube correspondingly and driven to generate heat by a pulse current input to the heating source through the driving circuit. Then liquid in the infusion tube is heated by heat conduction so that a temperature of the liquid is increased quickly. A driving signal of the heating source is periodic pulse current F(t) for periodically inputting heat Q₀(t) into the liquid in the infusion tube at a heating point of the infusion tube.

The measurement and analysis module for average velocity in infusion tube includes temperature sensors while a measurement principle and an analysis method are as the below. An inner diameter and an outer diameter of the infusion tube are respectively Rᵢ and Rₒ (both far more smaller than a characteristic length L). A density ρ_{w}, specific heat capacity c_{w}, and coefficient of thermal conductivity k_{w} of a material for a tube wall are all constants. A density ρ_{f}, specific heat capacity c_{f}, and coefficient of thermal conductivity k_{f} of the liquid being infused are also constants. The heat periodically input into the liquid medicine in the infusion tube at the heating point of the infusion tube by the dynamic heating module is Q₀ (t), and heat transferred to a point A of the infusion tube is Q_{A}(t). Set up a cylindrical coordinate system including z-axis in the longitudinal direction, r-axis in the radial direction, and θ-axis in the angular direction while the point A is set as an origin of the coordinate (z=0). Dynamic waveforms with a temperature of T_{(z,t)} are created in the tube when the heat Q_{A}(t) is transferred in the tube wall and the liquid in the tube and the dynamic waveforms satisfy the following thermal dispersion equation:

$$\frac{\partial T}{\partial t} + U \frac{\partial T}{\partial z} = α \frac{{\partial }^{2} T}{\partial {z}^{2}} \qquad ( 1 )$$

wherein U in the above equation satisfies the following equation:

$$\frac{U}{V} = {( 1 + \frac{1 - {ϕ}_{f}}{{ϕ}_{f}} β )}^{- 1} \qquad ( 2 )$$

wherein V is average velocity in an infusion tube going to be measured while β and ϕ_{f} satisfy the following equations:

$$β = \frac{{ρ}_{w} {c}_{w}}{{ρ}_{f} {c}_{f}} \qquad ( 3 )$$

$${ϕ}_{f} = {( {R}_{i} / {R}_{o} )}^{2} \qquad ( 4 )$$

wherein the α in the equation (1) satisfies the following equation:

$$α = {α}_{f} ( {ω}^{M} + \frac{P {e}^{2}}{48} {ω}^{C} ) \qquad ( 5 )$$

$$wherein , P e = \frac{V {R}_{i}}{{α}_{f}} \qquad ( 6 )$$

$$and {α}_{f} = \frac{{k}_{f}}{{ρ}_{f} {c}_{f}} \qquad ( 7 )$$

$${ω}^{M} ( {ϕ}_{f} , β , γ ) = \frac{{ϕ}_{f} + ( 1 - {ϕ}_{f} ) γ}{{ϕ}_{f} + ( 1 - {ϕ}_{f} ) β} \qquad ( 8 )$$

$$γ = \frac{{k}_{w}}{{k}_{f}} \qquad ( 9 )$$

$${ω}^{C} ( {ϕ}_{f} , β , γ ) = \frac{{ϕ}_{f}}{{[ {ϕ}_{f} + ( 1 - {ϕ}_{f} ) β ]}^{3}} \times [ \text{} {ϕ}_{{f}^{2}} + 6 {ϕ}_{f} ( 1 - {ϕ}_{f} ) β + 11 {( 1 - {ϕ}_{f} )}^{2} {β}^{2} - 6 \frac{{β}^{2}}{γ} \{ ( 1 - {ϕ}_{f} ( 3 - {ϕ}_{f} ) + 2 ln {ϕ}_{f} \} ] \qquad ( 10 )$$

when the heat at the point A of the infusion tube is Q_{A} (t), average temperature at the point A is T_{A}(t) and boundary conditions of the equation (1) satisfy the following equations:

$$T ( 0 , t ) = {T}_{A} ( t ) = {T}_{A} ( t + τ ) \qquad ( 11 a )$$

$$\frac{\partial T ( z , t )}{\partial t} \to 0 ( when z \to \pm ∞ , ∀ t ) \qquad ( 11 b )$$

By Fourier series expansion and separation of variables, a solution to a transfer function H(jω, z) in frequency domain is found according to the equations (1), (11a), and (11b) and the transfer function H(jω,z) in the frequency domain is represented as the following equation:

$$H ( j ω , z ) = {\tilde{T}}_{A} ( j ω ) / {\tilde{T}}_{z} ( j ω , z ) = exp [ \frac{z}{2 α} ( U - \sqrt{{U}^{2} - 4 j ω α} ) ] \qquad ( 12 )$$

wherein T_{A}(jω)) is a harmonic component of the average temperature at the point A T_{A} (t) corresponding to angular frequency ω and T_{B}(jω, z) is a harmonic component of the average temperature at any place z T_{z}(z,t) corresponding to the angular frequency ω.

The point A and point B having a distance L therebetween are both provided with a thermopile infrared temperature sensor for synchronous measurement of T_{A} (t) and T_{B}(L,t) to get a magnitude-frequency curve and a phase-frequency curve of a transfer function H(jω, L) in frequency domain. According to the equation (12), use least squares method for fitting the magnitude-frequency curve and the phase-frequency curve of the transfer function H(jω, L) respectively and both can get the U. The average velocity V of the liquid flowing in the tube is further obtained according to the equation (2).

The velocity adjustment module consists of a stopping plate with a recess and a lifting stepper motor. The infusion tube is arranged between the stopping plate with the recess and the lifting stepper motor. A cross sectional area S(d) of the infusion tube is adjusted by a stroke length d of the lifting stepper motor and the velocity is further adjusted. The velocity adjustment module is combined with the measurement and analysis module for average velocity in infusion tube and the micro control module to form a feedback system for precise velocity control.

The alarming and automatic clipping module is provided with an active buzzer for automatic alarm while the infusion liquid medicine runs out.

The mobile-phone-computer remote monitoring module includes a Bluetooth module and a user's end with a mobile phone or a computer. Another Bluetooth module in equipment of the user's end such as the mobile phone or the computer can be paired with the Bluetooth module to achieve human-computer interaction (HCI) by serial communication.

The operable shared module allows users to share and capture ID at user's end conveniently.

The micro control module is respectively connected with the dynamic heating module, the measurement and analysis module for average velocity in infusion tube, the velocity adjustment module, the alarming and automatic clipping module, and the mobile-phone-computer remote monitoring module. The micro control module provides the following functions.
A. Users can send commands of heating temperature and liquid velocity to the micro control module through the user's end. Then the micro control module controls respective modules to work and sends information monitored and obtained including temperature, velocity, and alarm back to the user's end to be displayed in a real-time manner.
B. An original key is stored in the micro control module. The user inputs a device ID through the mobile phone or computer at the user's end and by the operable shared module. Then the user's end gets and analyzes a key from the device and delivers the key to the micro control module by the Bluetooth module after completing analysis. The micro control module checks whether the key analyzed is identical to the original key. If they are identical, the micro control module provides feedback to the mobile-phone-computer remote monitoring module to get access to the device.
C. The micro control module checks whether an alarm is required according to average velocity V in the infusion tube. Once the micro control module confirms that the liquid medicine in a bottle is empty, it not only controls the active buzzer to send an alarm but also drives the lifting stepper motor to move upward to a bottom of the recess of the stopping plate for automatic clipping and closing of the infusion tube.

Moreover, three working modes of the system including a velocity measurement mode, a constant temperature heating mode, and a velocity control mode are set up in the mobile-phone-computer remote monitoring module and able to work together at the same time, or work independently.
(1) Under the velocity measurement mode and/or the velocity control mode, the micro control module checks whether the liquid medicine in the bottle is empty according to the average velocity V of the liquid flowing in the infusion tube obtained by the measurement and analysis module for average velocity in infusion tube.
(2) While working under the constant temperature heating mode, the driving signal of the dynamic heating module and velocity pulse current are superposed. The measurement and analysis module for average velocity in infusion tube measures the velocity and sends results to the micro control module for checking whether the liquid medicine in the bottle is empty.
(3) When none of the working modes is selected, the micro control module activates the dynamic heating module and the measurement and analysis module for average velocity in infusion tube at a specific time through an internal timer. The measurement and analysis module for average velocity in infusion tube sends calculation results to the micro control module to check whether the liquid medicine in the bottle is empty.

Furthermore, the heating source is a miniature ceramic heating sheet and the drive circuit is formed by metal-oxide-semiconductor field-effect transistor (MOSFET).

The present system has the following advantages.
1. The liquid medicine is automatically heated to body temperature so that patient's will not feel uncomfortable because of temperature difference between the liquid medicine and the body.
2. By real-time monitoring of the velocity of the infused liquid at the user's end, an alarm is issued and the infusion tube is clipped and closed automatically for protection of the patients when an abnormal situation is detected.
3. Medical staff can adjust the velocity of the infused liquid at the user's end according to the patient's physiological characteristics and further improve their work efficiency.
4. The system with simple structure and light weight can be directly clipped on the infusion tube.
5. The system which features on low cost, high precision, and easy operation can be produced into a shared device for promotion of smart medical care.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

FIG. 1 is a schematic drawing showing system architecture of an embodiment in which component 1 is a dynamic heating module, component 2 is a measurement and analysis module for average velocity in infusion tubes, component 3 is a velocity adjustment module, component 4 is an alarming and automatic clipping module, component 5 is a mobile-phone-computer remote monitoring module, component 6 is an operable shared module, and component 7 is a micro control module according to the present invention;

FIG. 2 is a schematic drawing showing geometric structure and a cylindrical coordinate system of an infusion tube of an embodiment according to the present invention;

FIG. 3 is a schematic drawing showing a velocity adjustment module of an embodiment according to the present invention;

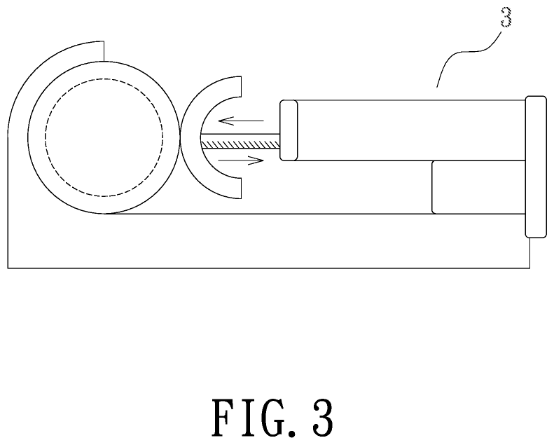

FIG. 4 shows waveforms of average temperature T_{A} (t) and T_{B}(L,t) at positions where two thermopile infrared temperature sensors are disposed and having a distance L of 33 mm therebetween obtained by numerical simulation of an embodiment according to the present invention;

FIG. 5 is a magnitude-frequency curve (solid-line are fitted values and dots are numerical simulation values) of a transfer function obtained by two thermopile infrared temperature sensors with a distance L of 33 mm therebetween of an embodiment according to the present invention;

FIG. 6 is a phase-frequency curve (solid-line are fitted values and dots are numerical simulation values) of a transfer function of obtained by two thermopile infrared temperature sensors with a distance L of 33 mm therebetween of an embodiment according to the present invention;

FIG. 7 shows waveforms of average temperature T_{A} (t) and T_{B}(L,t) at positions where two thermopile infrared temperature sensors are disposed and having a distance L of 33 mm therebetween obtained by actual measurement;

FIG. 8 is a magnitude-frequency curve (solid-line are fitted values and dots are measured values) of a transfer function obtained by two thermopile infrared temperature sensors with a distance L of 33 mm therebetween of an embodiment according to the present invention;

FIG. 9 is a phase-frequency curve (solid-line are fitted values and dots are measured values) of a transfer function of obtained by two thermopile infrared temperature sensors with a distance L of 33 mm therebetween of an embodiment according to the present invention.

### DETAILED DESCRIPTION OF THE PREFERRED EMBODIMENT

In order to learn technical content, purposes and functions of the present invention more clearly and completely, please refer to the following detailed descriptions with the figures and reference signs.

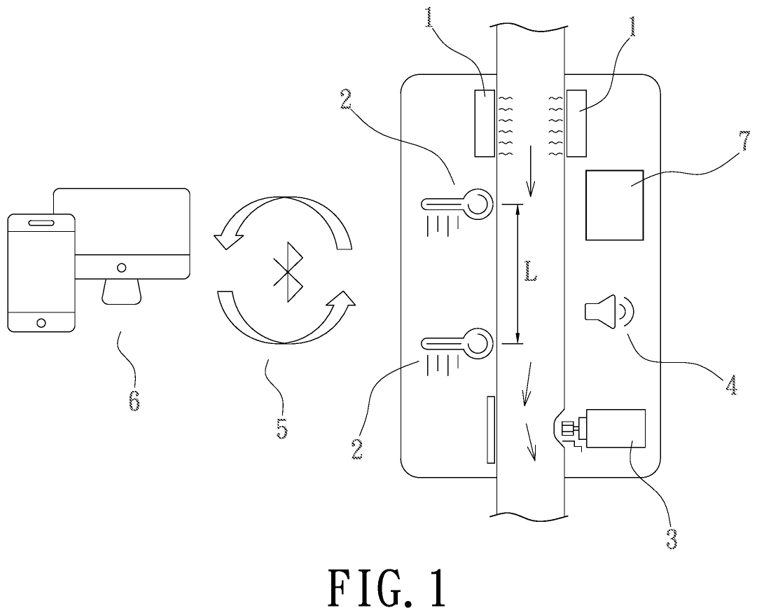

Refer to FIG. 1, an infusion liquid heating and flow-velocity-monitoring system for clinical use according to the present invention includes a dynamic heating module 1, a measurement and analysis module for average velocity in infusion tubes 2, a velocity adjustment module 3, an alarming and automatic clipping module 4, a mobile-phone-computer remote monitoring module 5, an operable shared module 6, and a micro control module 7.

The dynamic heating module 1 consists of heating sources and a driving circuit for the heating sources. The two heating sources are fixed on two sides of an infusion tube correspondingly and driven to generate heat by a pulse current input to the heating source through the driving circuit. Then liquid in the infusion tube is heated by heat conduction so that a temperature of the liquid is increased quickly. A driving signal of the heating source is periodic pulse current F(t) for periodically inputting heat Q₀(t) into the liquid such as liquid medicine in the infusion tube at a heating point of the infusion tube.

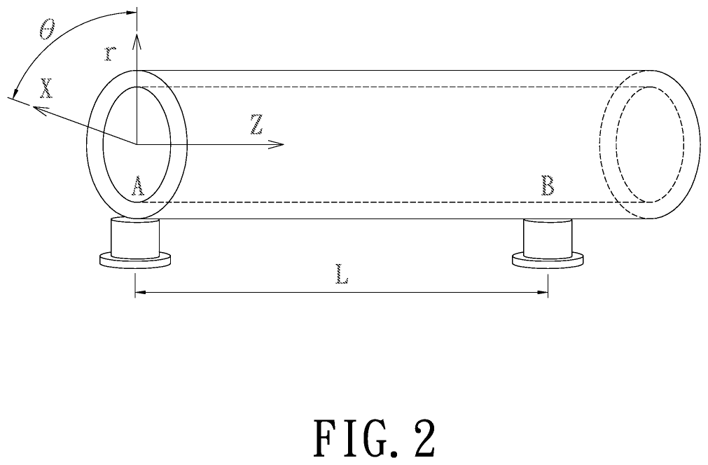

The measurement and analysis module for average velocity in infusion tube 2 includes temperature sensors and a method of measurement and analysis is as the below. As shown in FIG. 2, an inner diameter and an outer diameter of the infusion tube are respectively Rᵢ and Rₒ (both far more smaller than a characteristic length L). A density ρ_{w}, specific heat capacity c_{w}, and coefficient of thermal conductivity k_{w} of a material for a tube wall are all constants. A density ρ_{f}, specific heat capacity c_{f}, and coefficient of thermal conductivity k_{f} of the infusion liquid are also constants. The heat periodically input into the liquid medicine in the infusion tube at the heating point of the infusion tube by the dynamic heating module 1 is Q₀ (t), and heat transferred to a point A of the infusion tube is Q_{A} (t). As shown in FIG. 2, set up a cylindrical coordinate system including z-axis in the longitudinal direction, r-axis in the radial direction, and θ-axis in the angular direction while the point A is set as an origin of the coordinate (z=0). Dynamic waveforms with a temperature of T_{(z,t)} are created in the tube when heat Q_{A}(t) is transferred in the tube wall and the liquid in the tube and the dynamic waveforms satisfy the following thermal dispersion equation:

$$\frac{\partial T}{\partial t} + U \frac{\partial T}{\partial z} = α \frac{{\partial }^{2} T}{\partial {z}^{2}} \qquad ( 1 )$$

wherein U in the above equation satisfies the following equation:

$$\frac{U}{V} = {( 1 + \frac{1 - {ϕ}_{f}}{{ϕ}_{f}} β )}^{- 1} \qquad ( 2 )$$

wherein V is average velocity in an infusion tube going to be measured while β and ϕ_{f} satisfy the following equations:

$$β = \frac{{ρ}_{w} {c}_{w}}{{ρ}_{f} {c}_{f}} \qquad ( 3 )$$

$${ϕ}_{f} = {( {R}_{i} / {R}_{o} )}^{2} \qquad ( 4 )$$

wherein the α in the equation (1) satisfies the following equation;

$$α = {α}_{f} ( {ω}^{M} + \frac{P {e}^{2}}{48} {ω}^{C} ) \qquad ( 5 )$$

$$wherein P e = \frac{V {R}_{i}}{{α}_{f}} \qquad ( 6 )$$

$$and {α}_{f} = \frac{{k}_{f}}{{ρ}_{f} {c}_{f}} \qquad ( 7 )$$

$${ω}^{M} ( {ϕ}_{f} , β , γ ) = \frac{{ϕ}_{f} + ( 1 - {ϕ}_{f} ) γ}{{ϕ}_{f} + ( 1 - {ϕ}_{f} ) β} \qquad ( 8 )$$

$$γ = \frac{{k}_{w}}{{k}_{f}} \qquad ( 9 )$$

$${ω}^{C} ( {ϕ}_{f} , β , γ ) = \frac{{ϕ}_{f}}{{[ {ϕ}_{f} + ( 1 - {ϕ}_{f} ) β ]}^{3}} \times [ \text{} {ϕ}_{{f}^{2}} + \text{} 6 {ϕ}_{f} ( 1 - {ϕ}_{f} ) β + 11 {( 1 - {ϕ}_{f} )}^{2} {β}^{2} - 6 \frac{{β}^{2}}{γ} \{ ( 1 - {ϕ}_{f} ) ( 3 - {ϕ}_{f} ) + 2 ln {ϕ}_{f} \} ] \qquad ( 10 )$$

As shown in FIG. 1, when the heat at the point A of the infusion tube is Q_{A} (t), average temperature at the point A is T_{A} (t) and boundary conditions of the equation (1) satisfy the following equations:

$$T ( 0 , t ) = {T}_{A} ( t ) = {T}_{A} ( t + τ ) \qquad ( 11 a )$$

$$\frac{\partial T ( z , t )}{\partial t} \to 0 ( when z \to \pm ∞ , ∀ t ) \qquad ( 11 b )$$

By Fourier series expansion and separation of variables, a solution to a transfer function H(jω, z) in frequency domain is found according to the equations (1), (11a), and (11b) and the transfer function H(jω, z) in the frequency domain is represented as the following equation:

$$H ( j ω , z ) = {\tilde{T}}_{A} ( j ω ) / {\tilde{T}}_{z} ( j ω , z ) = exp [ \frac{z}{2 α} ( U - \sqrt{{U}^{2} - 4 j ω α} ) ] \qquad ( 12 )$$

wherein T_{A}(jω) is a harmonic component of the average temperature at the point A T_{A} (t) corresponding to angular frequency ω and T_{B} (jω, z) is a harmonic component of the average temperature at any place z T_{z}(z,t) corresponding to the angular frequency ω.

The point A and point B having the distance L therebetween are both provided with a thermopile infrared temperature sensor for synchronous measurement of the T_{A} (t) and T_{B}(L,t) to get a magnitude-frequency curve and a phase-frequency curve of the transfer function H(jω, L) in the frequency domain. According to the equation (12), use least squares method for fitting the magnitude-frequency curve and the phase-frequency curve of the transfer function H(jω, L) respectively and both can get the U. The average velocity V of the liquid flowing in the tube is further obtained according to the equation (2).

The velocity adjustment module 3 consists of a stopping plate with a recess and a lifting stepper motor. The infusion tube is arranged between the stopping plate with the recess and the lifting stepper motor. A cross sectional area S(d) of the infusion tube is adjustable by a stroke length d of the lifting stepper motor and the velocity is further adjusted. The velocity adjustment module 3 is combined with the measurement and analysis module for average velocity in infusion tube 2 and the micro control module 7 to form a feedback system for precise velocity adjustment.

The alarming and automatic clipping module 4 is provided with an active buzzer for automatic alarm while the liquid medicine runs out.

The mobile-phone-computer remote monitoring module 5 includes a Bluetooth module and a user's end with a mobile phone or a computer. Another Bluetooth module in equipment of the user's end such as the mobile phone or the computer can be paired with the Bluetooth module to achieve human-computer interaction (HCI) by serial communication.

The operable shared module 6 allows users to share and capture ID at the user's end conveniently.

The micro control module 7 is respectively connected with the dynamic heating module 1, the measurement and analysis module for average velocity in infusion tube 2, the velocity adjustment module 3, the alarming and automatic clipping module 4, and the mobile-phone-computer remote monitoring module 5 and having the functions below.
A. Users can send commands related to heating temperature and liquid velocity to the micro control module 7 through the user's end. Then the micro control module 7 controls respective modules to work and sends information monitored and obtained including temperature, velocity, and alarm back to the user's end to be displayed in a real-time manner.
B. An original key is stored in the micro control module 7. The user inputs a device ID through the mobile phone or computer at the user's end and by the operable shared module 6. Then the user's end gets and analyzes a key from the device and delivers the key to the micro control module 7 by the Bluetooth module after completing analysis. The micro control module 7 checks whether the key analyzed is identical to the original key. If they are identical, the micro control module 7 provides feedback to the mobile-phone-computer remote monitoring module 5 to get access to the device.
C. The micro control module 7 checks whether an alarm is required according to average velocity V in the infusion tube. Once the micro control module 7 checks that the liquid medicine in a bottle is empty, it controls the active buzzer to send an alarm and drives the lifting stepper motor to move upward to a bottom of the recess of the stopping plate for automatic clipping and closing of the infusion tube.

Moreover, the mobile-phone-computer remote monitoring module 5 is provided with three working modes of the system including a velocity measurement mode, a constant temperature heating mode, and a velocity control mode, which are able to work together at the same time, or work independently.
(1) While working at the velocity measurement mode and/or the velocity control mode, the micro control module 7 checks whether the liquid medicine in the bottle is empty according to the average velocity V of the liquid flowing in the infusion tube obtained by the measurement and analysis module for average velocity in infusion tube 2.
(2) While working at the constant temperature heating mode, the driving signal of the dynamic heating module 1 and velocity pulse current are superposed. The measurement and analysis module for average velocity in infusion tube 2 measures the velocity and sends results to the micro control module 7 for checking whether the liquid medicine in the bottle is empty.
(3) When none of the working modes is selected, the micro control module 7 activates the dynamic heating module 1 and the measurement and analysis module for average velocity in infusion tube 2 at a specific time through an internal timer. The measurement and analysis module for average velocity in infusion tube 2 sends calculation results to the micro control module 7 to check whether the liquid medicine in the bottle is empty.

Furthermore, the heating source is a miniature ceramic heating sheet and the drive circuit is formed by metal-oxide-semiconductor field-effect transistor (MOSFET).

For further explanation of the present invention, please refer to the following embodiments, but not to limit the scope of the present invention.
(1) The micro control module 7 used in the present invention is STM32F103C8T6 minimum system which is capable of completing control, calculation, and analysis of the respective modules. A size of the miniature ceramic heating sheet in the dynamic heating module 1 is 10 mm×10 mm for better attachment to the infusion tube. In the measurement and analysis module for average velocity in infusion tube 2, a XGZT263 thermopile infrared temperature sensor used has advantages of quick response, low cost, and high precision within range around human body temperature. In order to clip and close the infusion tube completely, the stopping plate with the recess in the velocity adjustment module 3 is attached closely to an inner wall of the infusion tube and a top end of the lifting stepper motor is also designed into a recess form. The alarming and automatic clipping module 4 includes the active buzzer to which the micro control module 7 sends a trigger signal for alarming. The Bluetooth module of the mobile-phone-computer remote monitoring module 5 uses low power chip CH9140 which provide transmission within a hundred-meter radius and Qt software is used at the user's end.
(2) A housing for the device is produced. The housing including a cylindrical hole in a middle part for mounting the infusion tube. Place two miniature ceramic heating sheets on two sides of an upper part/stream of the infusion tube correspondingly. Then arrange the measurement and analysis module for average velocity in infusion tube 2. Along flowing direction of the liquid medicine in the infusion tube, put the first thermopile infrared temperature sensor on the infusion tube at the point A which is 20 mm far away from the miniature ceramic heating sheet. Next dispose the second thermopile infrared temperature sensor on the infusion tube at the point B which is 33 mm moved backward from the point A. Lastly fix the lifting stepper motor on the stopping plate located at a rear end of the housing and aligned with the recess.
(3) Turn on the user' end which includes a login interface, a data display interface, and a control interface. The Bluetooth module built in the equipment of the user' end and the Bluetooth module inside the device (system) are paired first. After entering the device ID through the login interface, the user's end gets and analyzes a key from the device and delivers the key to the micro control module 7 by the Bluetooth module after completing analysis. The micro control module 7 compares with the original key. If they are identical, the user can continue to operate the device and jump to the control interface to select at least one of the three working modes including the velocity measurement mode, the constant temperature heating mode, and the velocity control mode.
(4) Once the constant temperature heating mode is selected, set up heating temperature by the control interface. In order to avoid degradation of medicine caused by high temperature, the maximum temperature the user can set is unable to be over 37° C. The micro control module 7 sends a 1 KHz Pulse-width modulation (PWM) driving signal to the dynamic heating module 1 and then the driving circuit converts the driving signal to a corresponding pulse current for driving the miniature ceramic heating sheet to generate heat. The thermopile infrared temperature sensor at the point B of the measurement and analysis module for average velocity in infusion tube 2 detects temperature information which is then sent back to the micro control module 7 for comparison with a set value. A duty ratio of the PWM driving signal is modulated by proportional—integral—derivative (PID) algorism to provide feedback control for precise velocity adjustment. At last, the micro control module 7 sends the heating temperature to the data display interface of the user's end.
(5) If the velocity measurement mode is selected, the driving signal of the dynamic heating module 1 and a 0.02 Hz, 30% duty ratio velocity-measurement driving signal are superposed by the micro control module 7 and the driving circuit converts the driving signal to a corresponding pulse current for driving the miniature ceramic heating sheet to generate heat. The measurement and analysis module for average velocity in infusion tube 2 detects temperature of the flowing liquid medicine and gets the average velocity V in the tube after analysis and calculation. Then the velocity is delivered to the user's end by the mobile-phone-computer remote monitoring module 5 for real-time display.
(6) When the velocity measurement mode is selected, first set up velocity expected through the user's end. The micro control module 7 gets the average velocity V by the velocity measurement mode and then compares the velocity V with the set value. Use the PID algorism to drive the lifting stepper motor of the velocity adjustment module 3 and adjust the stroke length d. Thereby the feedback control is provided for precise velocity adjustment.
(7) The micro control module 7 uses its internal timer to monitor the velocity once per 2 minutes and a velocity measurement mode is added besides the original working modes for checking conditions of the liquid medicine in the bottle. Once an abnormal situation is detected, the micro control module 7 not only issues an alarm by sending the trigger signal to the active buzzer of the alarming and automatic clipping module 4 for alarming but also drives the lifting stepper motor to clip and close the infusion tube automatically.
(8) Implementation of the measurement and analysis module for average velocity in infusion tube 2: an inner diameter and an outer diameter of the infusion tube for medical use now are respectively 1.5 mm and 2 mm; a material for the tube wall having the density ρ_{w} of 1390 kgm⁻³, specific heat capacity c_{w} of 1050 Jkg⁻¹K⁻¹, and coefficient of thermal conductivity k_{w} of 0.17 Wm⁻¹K⁻¹; the density ρ_{f}, specific heat capacity c_{f}, and coefficient of thermal conductivity k_{f} of the infusion liquid are respectively 1000 kgm⁻³, 4200 Jkg⁻¹K⁻¹, and 0.59 Wm⁻¹K⁻¹; after a control system getting the command from a master computer. the miniature ceramic heating sheet is driven by the pulse current F(t) for periodically inputting heat Q(t) into the heating point of the infusion tube and the heat Q(t) is transferred in the tube wall and the liquid in the infusion tube. After periodic waveforms being stabilized, the average temperatures T_{A}(t) and T_{B} (L,t) are synchronously measured and recorded by the thermopile infrared temperature sensors at the point A and the point B with the distance L of 33 mm therebetween and a solution to a transfer function H(jω, z) in frequency domain is obtained according to the equations (1), (11a), and (11b) by Fourier series expansion and separation of variables. The average velocity V of the liquid in the tube can be obtained according to the equation (2) by the already-known distance L between the two thermopile infrared temperature sensors.

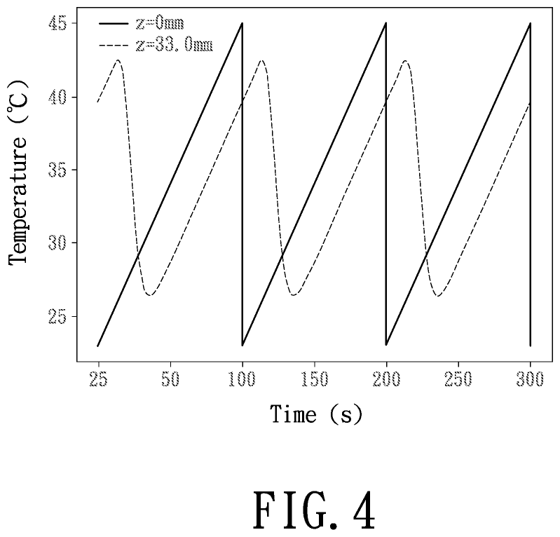

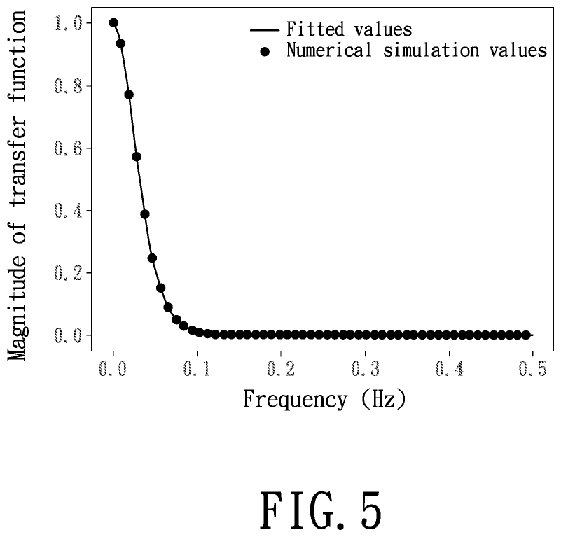

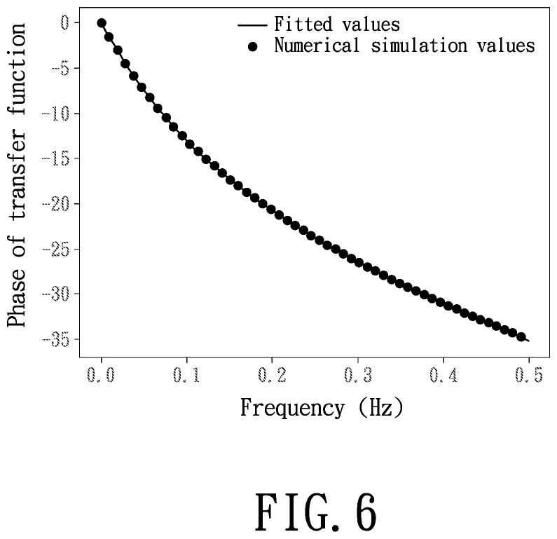

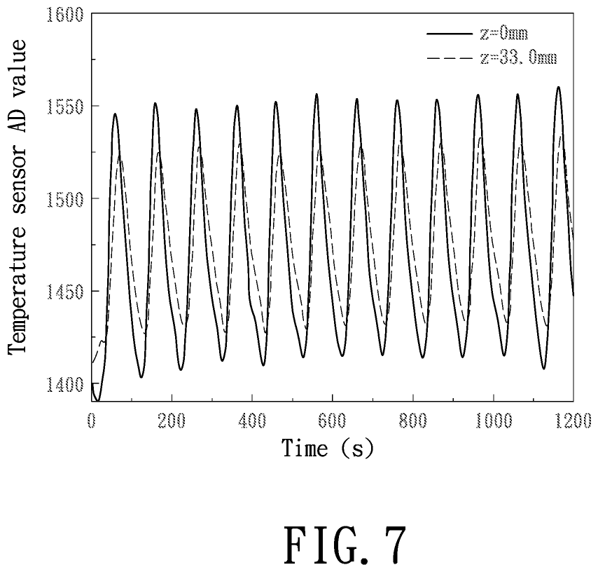

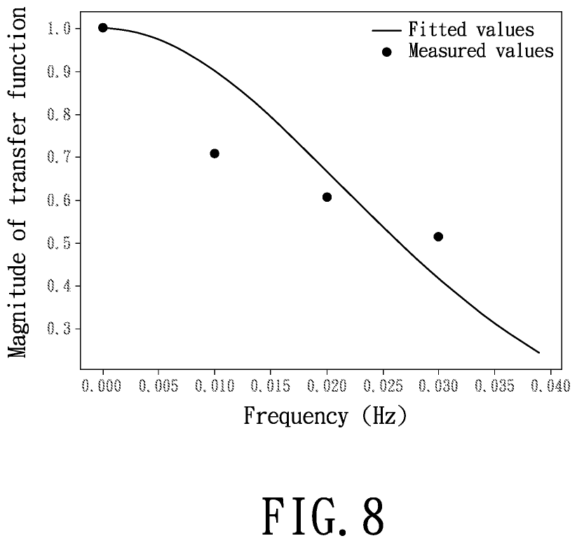

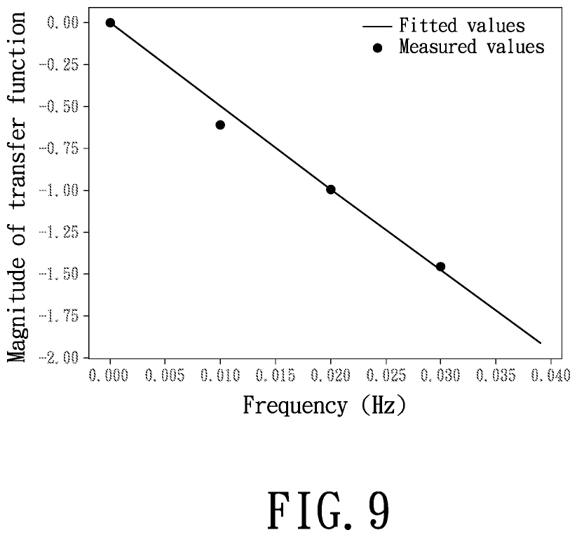

In order to validate effectiveness of the velocity measurement method, the present invention use two methods, numerical simulation and experimental measurement.
1. Numerical simulation method: it is assumed that the average velocity in the infusion tube is 2.12 mm/s. As shown in FIG. 4, the average temperature T_{A}(t) and T_{B}(L,t) are obtained by numerical simulation of the equations (1), (11a), and (11b) and then a magnitude-frequency curve of the transfer function (as shown in FIG. 5) and a phase-frequency curve (as shown in FIG. 6) of the transfer function are further obtained. Use the least squares method for fitting the magnitude-frequency curve (FIG. 5) to get the average velocity V of the flowing liquid in the tube, 2.14 mm/s (relative error is 0.94% when the true value is 2.12 mm/s). Also use the least squares method for fitting the phase-frequency curve (FIG. 6) to get the average velocity V of the flowing liquid in the tube, 2.08 mm/s (relative error is 1.87% when the true value is 2.12 mm/s).
2. Experimental measurement method: as shown in FIG. 7, the average temperature T_{A} (W and T_{B} (L,t) are measured and a magnitude-frequency curve of the transfer function (as shown in FIG. 8) and a phase-frequency curve (as shown in FIG. 9) of the transfer function are further obtained. There is a huge error when the magnitude-frequency curve (FIG. 8) is compared with the actual value because the two temperature sensors in the design have different sensitivities. Thus use the least squares method for fitting the phase-frequency curve (FIG. 9) to get the average velocity V of the flowing liquid in the tube, 5.24 mm/s.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalent.

What is claimed is:

1. An infusion liquid heating and flow-velocity-monitoring system for clinical use comprising a dynamic heating module, a measurement and analysis module for average velocity in infusion tubes, a velocity adjustment module, an alarming and automatic clipping module, a mobile-phone-computer remote monitoring module, an operable shared module, and a micro control module;
wherein the dynamic heating module includes at least two heating sources and at least one driving circuit for the heating sources; the two heating sources are fixed on two sides of an infusion tube correspondingly and driven to generate heat by a pulse current input to the heating source through the driving circuit; a liquid in the infusion tube is heated by heat conduction so that a temperature of the liquid is increased quickly; a driving signal of the heating source is periodic pulse current F(t) for periodically inputting heat Q₀(t) into the liquid in the infusion tube at a heating point of the infusion tube;
wherein the measurement and analysis module for average velocity in infusion tube includes temperature sensors while a measurement principle and an analysis method are as the following: an inner diameter and an outer diameter of the infusion tube are respectively Rᵢ and Rₒ, both far more smaller than a characteristic length L; a density ρ_{w}, specific heat capacity c_{w}, and coefficient of thermal conductivity k_{w} of a material for a tube wall of the infusion tube are constants; a density ρ_{f}, specific heat capacity c_{f}, and coefficient of thermal conductivity k_{f} of the liquid being infused are constants; the heat periodically input into the liquid in the infusion tube at the heating point of the infusion tube by the dynamic heating module is Q₀(t), and heat transferred to a point A of the infusion tube is Q_{A}(t); set up a cylindrical coordinate system including z-axis in the longitudinal direction of the infusion tube, r-axis in the radial direction of the infusion tube, and θ-axis in the angular direction of the infusion tube while the point A is set as an origin of the coordinate, z=0; dynamic waveforms with a temperature of T_{(z,t)} are created in the tube when the heat Q_{A}(t) is transferred in the tube wall and the liquid in the infusion tube and the dynamic waveforms satisfy the following thermal dispersion equation:
$$\frac{\partial T}{\partial t} + U \frac{\partial T}{\partial z} = α \frac{{\partial }^{2} T}{\partial {z}^{2}} \qquad ( 1 )$$
wherein U in the above equation satisfies the following equation:
$$\frac{U}{V} = {( 1 + \frac{1 - {ϕ}_{f}}{{ϕ}_{f}} β )}^{- 1} \qquad ( 2 )$$
wherein V is average velocity in an infusion tube going to be measured while β and ϕ_{f} satisfy the following equations:
$$β = \frac{{ρ}_{w} {c}_{w}}{{ρ}_{f} {c}_{f}} \qquad ( 3 )$$
$${ϕ}_{f} = {( {R}_{i} / {R}_{o} )}^{2} \qquad ( 4 )$$
wherein the α in the equation (1) satisfies the following equation:
$$α = {α}_{f} ( {ω}^{M} + \frac{P {e}^{2}}{48} {ω}^{C} ) \qquad ( 5 )$$
$$wherein P e = \frac{V {R}_{i}}{{α}_{f}} \qquad ( 6 )$$
$$and {α}_{f} = \frac{{k}_{f}}{{ρ}_{f} {c}_{f}} \qquad ( 7 )$$
$${ω}^{M} ( {ϕ}_{f} , β , γ ) = \frac{{ϕ}_{f} + ( 1 - {ϕ}_{f} ) γ}{{ϕ}_{f} + ( 1 - {ϕ}_{f} ) β} \qquad ( 8 )$$
$$γ = \frac{{k}_{w}}{{k}_{f}} \qquad ( 9 )$$
$${ω}^{C} ( {ϕ}_{f} , β , γ ) = \frac{{ϕ}_{f}}{{[ {ϕ}_{f} + ( 1 - {ϕ}_{f} ) β ]}^{3}} \times [ \text{} {ϕ}_{f}^{2} + 6 {ϕ}_{f} ( 1 - {ϕ}_{f} ) β + 11 {( 1 - {ϕ}_{f} )}^{2} {β}^{2} - 6 \frac{{β}^{2}}{γ} \{ ( 1 - {ϕ}_{f} ) ( 3 - {ϕ}_{f} ) + 2 ln {ϕ}_{f} \} ] \qquad ( 10 )$$
when the heat at the point A of the infusion tube is Q_{A} (t), average temperature at the point A is T_{A} (t) and boundary conditions of the equation (1) satisfy the following equations:
$$T ( 0 , t ) = {T}_{A} ( t ) = {T}_{A} ( t + τ ) \qquad ( 11 a )$$
$$\frac{\partial T ( z , t )}{\partial t} \to 0 ( when z \to \pm ∞ , ∀ t ) \qquad ( 11 b )$$
By Fourier series expansion and separation of variables, a solution to a transfer function H(jω,z) in frequency domain is found
according to the equations (1), (11a), and (11b) and the transfer function H(jω,z) in the frequency domain is represented as the following equation:
$$H ( j ω , z ) = {\tilde{T}}_{A} ( j ω ) / {\tilde{T}}_{z} ( j ω , z ) = exp [ \frac{z}{2 α} ( U - \sqrt{{U}^{2} - 4 j ω α} ) ] \qquad ( 12 )$$
wherein T_{A}(jω) is a harmonic component of the average temperature at the point A T_{A}(t) corresponding to angular frequency ω and T_{B}(jω, z) is a harmonic component of the average temperature at any place z T_{z}(z,t) corresponding to the angular frequency ω;
the point A and point B having a distance L therebetween are both provided with the thermopile infrared temperature sensor for synchronous measurement of T_{A}(t) and T_{B} (L,t) to get a magnitude-frequency curve and a phase-frequency curve of a transfer function H(jω,L) in frequency domain; according to the equation (12), use least squares method for fitting the magnitude-frequency curve and the phase-frequency curve of the transfer function H(jω,L) respectively and both can get the U; the average velocity V of the liquid flowing in the tube is further obtained according to the equation (2);
wherein the velocity adjustment module consists of a stopping plate with a recess and a lifting stepper motor; the infusion tube is arranged between the stopping plate with the recess and the lifting stepper motor; a cross sectional area S(d) of the infusion tube is adjusted by a stroke length d of the lifting stepper motor and the velocity is further adjusted; the velocity adjustment module is combined with the measurement and analysis module for average velocity in infusion tube and the micro control module to form a feedback system for precise velocity control;
wherein the alarming and automatic clipping module is provided with an active buzzer for automatic alarm while the liquid runs out;
wherein the mobile-phone-computer remote monitoring module includes a Bluetooth module and a user's end with a mobile phone or a computer;
another Bluetooth module in equipment of the user's end such as the mobile phone or the computer is paired with the Bluetooth module to achieve human-computer interaction (HCI) by serial communication;
wherein the operable shared module allows users to share and capture ID at the user's end conveniently;
wherein the micro control module is respectively connected with the dynamic heating module, the measurement and analysis module for average velocity in infusion tube, the velocity adjustment module, the alarming and automatic clipping module, and the mobile-phone-computer remote monitoring module; the micro control module provides functions of:
wherein the user send commands related to heating temperature and liquid velocity to the micro control module through the user's end and then the micro control module controls the dynamic heating module, the measurement and analysis module for average velocity in infusion tube, the velocity adjustment module, the alarming and automatic clipping module, and the mobile-phone-computer remote monitoring module respectively to work and sends information monitored and obtained including temperature, velocity, and alarm back to the user's end to be displayed in a real-time manner;
wherein an original key is stored in the micro control module and the user inputs a device ID through the mobile phone or computer at the user's end and by the operable shared module; then the user's end gets and analyzes a key from the device and delivers the key to the micro control module by the Bluetooth module after completing analysis; the micro control module checks whether the key analyzed is identical to the original key; if they are identical, the micro control module provides feedback to the mobile-phone-computer remote monitoring module to get access to the device;
wherein the micro control module checks whether an alarm is required according to average velocity in the infusion tube; once the micro control module confirms that the liquid in a bottle is empty, it not only controls the active buzzer to send an alarm but also drives the lifting stepper motor to move upward to a bottom of the recess of the stopping plate for automatic clipping and closing of the infusion tube.

2. The system as claimed in claim 1, wherein three working modes of the system including a velocity measurement mode, a constant temperature heating mode, and a velocity control mode are set up in the mobile-phone-computer remote monitoring module and able to work together simultaneously, or work independently;
while under the velocity measurement mode and/or the velocity control mode, the micro control module checks whether the liquid in the bottle is empty according to the average velocity V of the liquid flowing in the infusion tube obtained by the measurement and analysis module for average velocity in infusion tube;
while under the constant temperature heating mode, the driving signal of the dynamic heating module and velocity pulse current are superposed;
the measurement and analysis module for average velocity in infusion tube performs velocity measurement and sends results to the micro control module for checking whether the liquid in the bottle is empty;
when none of the working modes is selected, the micro control module activates the dynamic heating module and the measurement and analysis module for average velocity in infusion tube at a specific time through an internal timer; the measurement and analysis module for average velocity in infusion tube sends calculation results to the micro control module for checking whether the liquid in the bottle is empty.

3. The system as claimed in claim 1, wherein the heating source is a miniature ceramic heating sheet and the drive circuit is formed by metal-oxide-semiconductor field-effect transistor (MOSFET).
